(19) United States
(12) Patent Application Publication
(10) Pub. No.: US 2013/0178807 A1
(43) Pub. Date: Jul. 11, 2013

(54) NEEDLE TIP GUARD FOR INTRAVENOUS CATHETER ASSEMBLY

(71) Applicant: Rishi Baid, New Delhi (IN)

(72) Inventor: Rishi Baid, New Delhi (IN)

(73) Assignee: Poly Medicure Limited, Faridabad, Haryana (IN)

(21) Appl. No.: 13/825,352

(22) PCT Filed: Sep. 21, 2011
(86) PCT No.: PCT/IB11/54136

§ 371 Date: Mar. 21, 2013

(30) Foreign Application Priority Data
Sep. 21, 2010 (IN) 2252/DEL/2010

(51) Int. Cl. A61M 5/00

(52) U.S. Cl. CPC A61M 5/00
USPC 604/263

(57) ABSTRACT

A needle tip guard for an intravenous catheter assembly, including a base portion having a bore extending along an axial direction for receiving a needle having a needle shaft extending through the bore and moving relative to the needle tip guard from a spread-apart position; first and second jaws extending from the distal side of the base portion generally in the axial direction; the first and second jaws having head portions at a free end, said first jaw resting over the needle shaft in the spread-apart position; with locking means on the outer surface of the needle tip guard for securing the needle tip guard to the medical device; and at least one elastic element integrally mounted onto the jaws in a region between the base portion and the head portions exerting an inward restoring force on the jaws when the jaws are spread apart by the needle shaft.

### CROSS-REFERENCE TO THE RELATED APPLICATION

This application claims priority from Indian Patent Application No. 2252/DEL/2010, filed on Sep. 21, 2010, the entire disclosure of which is incorporated herein by reference.

### FIELD OF THE INVENTION

The present invention relates to a needle tip guard for medical device. More particularly, present invention relates to a needle tip guard for an intravenous catheter assembly.

### BACKGROUND OF THE INVENTION

Accidental needle sticks/pricks have become a major concern to health-care worker. It is well known and understood by healthcare workers that sharp surgical instruments, such as catheter needles, have a significant potential for harm to healthcare workers. During surgery, handling of these sharp instruments can lead to accidental sticks or puncture wounds exposing the healthcare worker to the infections such as AIDS and hepatitis. The chances of needle stick are increased during an emergency with several aspects require to be handled. Likewise, during disposal, an exposed needle point may be and usually is a threat to the medical waste handler.

Such needle tip guards are generally known and operative as a guard for the tip of a needle of the medical device by automatically covering the needle tip during withdrawal of the needle, for example, from a patient. The needle safety device thereby serves to prevent accidental pricking of, for example, a healthcare worker by the needle after removal of the needle from the medical device.

There is a need to protect the healthcare workers from accidental needle pricks. That being there is a constant need for having a needle tip guard to prevent accidental pricks and sticks.

### SUMMARY OF THE INVENTION

The invention relates to a needle tip guard for a medical device, in particular for an intravenous catheter assembly, the needle tip guard comprising: a base portion having a bore extending along an axial direction for receiving a needle having a needle shaft such that the needle may extend through the bore and move relative to the needle tip guard from a spread-apart position in which the needle tip is outside the needle tip guard to a protective position in which the needle tip is covered by the needle tip guard; first and second jaw extending from the distal side of the base portion generally in the axial direction; each of the said first and second jaws having a head portion in the region of its free end, wherein the said first jaw rests over the needle shaft in the spread-apart position; one or more locking means onto the outer surface of the needle tip guard for securing the said needle tip guard to the said medical device; and at least one resilient member or elastic element integrally mounted onto the said jaws in a region between the said base portion and the head portions such that said resilient member or elastic element exerts an inward restoring force on the said jaws, when the jaws are fully or partially spread apart by the said needle shaft.

Further features of the invention provides for the said resilient member or elastic element integrally mounted to mounting features provided on each of the jaws. The mounting features may comprise one or more mounting projections and/or mounting recesses.

Further features of the invention provides for the said resilient member or elastic element integrally mounted onto the jaws being adapted to form a partial sidewall such that together with the jaws it defines a chamber surrounding the needle in which the needle tip is held after complete withdrawal of the needle from the medical device once the said jaws snaps together protecting the needle tip.

Further features of the invention provides for the said integrated resilient member or elastic element to comprise a ring like integrated form/structure partially or fully surrounding the jaws, and/or clamp, bracket, “C” clip or the like surrounding the jaws only in part.

Further features of the invention provides for one or more locking and/or releasing means provided either or both inside and/or outside of the said catheter housing for engaging and/or releasing the said needle guard in the form of one or more projections and/or recesses. Likewise, the outer surface of the needle tip guard may be provided with one or more locking and/or releasing means capable of being in a locking and/or releasing arrangement with the catheter housing of the said medical device.

Another aspect of the invention relates to an intravenous catheter assembly, comprising a catheter tube, a catheter hub/housing, a needle having a needle tip, a needle shaft and a needle hub, and a needle tip guard as mentioned above slidably arranged on the needle for protecting the needle tip; and locking and/or releasing means formed inside and/or outside of the said catheter hub/housing for engaging and/or releasing the said needle guard.

It is, therefore, one of the objects of this invention to provide a needle tip guard which provides a better protection against accidental needle pricks/sticks reducing the complexity and costs involved in the manufacture of prior known needle tip guards.

Another object of the present invention is to ensure low cost fabrication, the number of injection and/or insert molded parts required for a needle safety portion of a device based upon the invention may be as few as one.

Yet another object of the present invention is that the medical device such as catheter assembly are stored and shelved prior to use with the jaws spread apart over the needle. Due to the integrated resilient member or elastic element in the needle tip guard exerting its restoring force on the jaws in the spread apart state of the jaws, the elastic element ensures that the spread apart jaws will snap together and guard the needle tip upon withdrawn of the needle from the medical device even after a longer shelf life, thereby continuously ensuring a correct functioning of the needle tip guard.

### BRIEF DESCRIPTION OF THE DRAWINGS

The present invention will now be explained in more detail in the following with reference to the preferred embodiments and to the accompanying drawings, wherein:

FIG. 1 illustrates a cross-section view of the needle tip guard according to the present invention;

FIG. 2 illustrates a side view of the needle tip guard according to the present invention;

FIG. 3 illustrates a front view of the needle tip guard according to the present invention;

FIG. 4 illustrates a perspective view of the needle tip guard according to the present invention;

FIG. 5 illustrates a top view of the needle tip guard according to the present invention;

FIG. 6 illustrates a bottom view of the needle tip guard according to the present invention;

FIG. 7A illustrates a side of the needle tip region of the needle according to the present invention;

FIG. 7B illustrates a plan view region of the needle tip region of FIG. 7A according to the present invention;

FIG. 8 illustrates a plan view of the catheter housing of the medical according to the present invention;

FIG. 9A illustrates a cross-sectional view of a section of the catheter housing of FIG. 8 detailing an annular locking protrusion;

FIG. 9B illustrates cross-sectional view of a section of the catheter housing of FIG. 8 detailing an alternative embodiment of the annular locking protrusion.

### DETAILED DESCRIPTION OF THE INVENTION

In accordance with the present invention, an improved needle tip guard for a medical device is provided. While this invention is susceptible of embodiments in many different forms, there will be described herein specific embodiments with the understanding that the present disclosure is to be considered as an exemplification of the principles of the invention and is not intended to limit the invention to the embodiments detailed herein.

As used herein, the term “proximal” refers to a region of the device or a location on the device which is closest to, for example, a clinician using the device. In contrast to this, the term “distal” refers to a region of the device which is farthest from the clinician, for example, the distal region of a needle will be the region of a needle containing the needle tip which is to be inserted e.g. into a patient's vein. The term “protective position” refers to the position in which the needle tip is inside the needle tip guard being covered by the jaws of the needle tip guard.

With reference to accompanying figures, when the needle 24 extends all the way through the needle tip guard 10, such as prior to use of the intravenous catheter device (not shown), the needle tip guard 10 is slidably arranged partially and/or completely in a chamber of a medical device, such as in the catheter housing 42 of an intravenous catheter apparatus, and positioned near a proximal end of the needle hub. In this situation, the needle 34 extends completely through the needle guard 10 and the distal end section 24 of the first jaw 16 is supported on the needle shaft 38, whereby the first jaw 16 is forced away from the second jaw 18 from its relaxed position into a spread-apart position. The term “spread-apart position” refers to the position in which the needle 34 having a needle shaft 38 passes through the jaws 16, 18 of the needle tip guard 10 such that the needle tip 36 is outside the needle tip guard 10. As opposed to this the “protective position” refers to the position in which the needle tip 36 is inside the needle tip guard 10 being covered by the jaws 16, 19 of the needle tip guard 10.

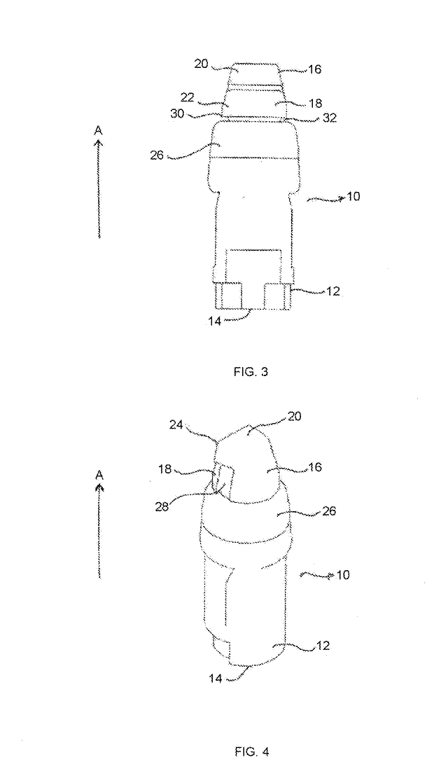

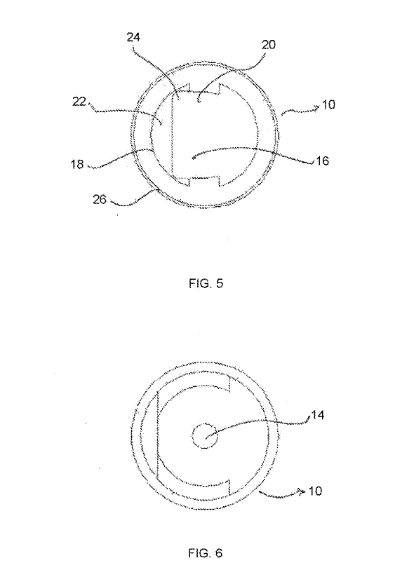

Referring to FIGS. 1 to 6, the needle tip guard 10 comprises a base portion 12 which has a bore 14 extending in the axial direction-A therethrough for receiving the needle 34 having a needle shaft 38 thereby defining a passage for the needle 34. The dimension of the bore 14 is adapted to the shape and size of the principal outer profile of the needle 34 such that the needle 34 may extend through the bore 14 and move relative to the needle tip guard 10 to a protective position in which the needle tip 36 is covered by the needle guard 10.

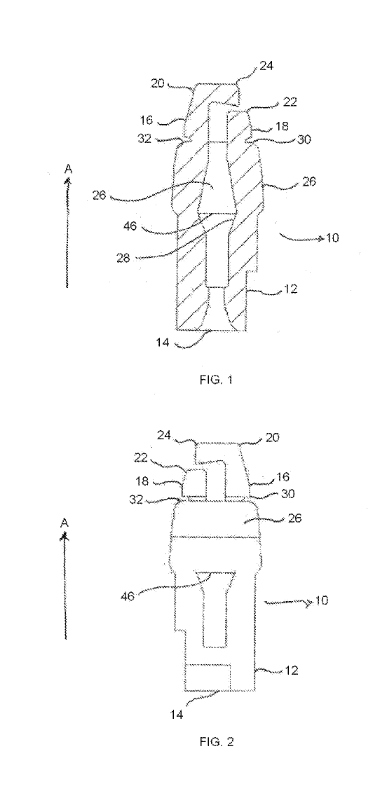

As shown in FIGS. 1 and 2, the needle tip guard 10 further includes first and second jaws 16, 18 extending distally from the base portion 12 generally in the axial direction A. The first jaw 16 has a first head portion 20 in the region of its free end, and the second jaw 18 has a second head portion 22 in the region of its free end. The first head portion 20 extends beyond the second head portion 22 and has a distal end section 24 at its free end, which extends towards the second jaw 18 in a direction generally perpendicular to the axial direction-A. The length of the distal end section 24 is as such that the end section 24 protrudes over at least a part of the second head portion 22, when the jaws 16, 18 are in a relaxed position as is shown in FIGS. 2 to 6. Once the needle tip 36 is protected completely within the needle tip guard 10, the protruding distal end section 24 abuts the tip to extend beyond the first head portion 20 thereby securing the tip 36 of the needle 34. For ease in understanding, the relaxed position refers to a situation where no needle 34 extends through the needle tip guard 10.

As is illustrated in FIGS. 1 to 4 the integrated resilient member or elastic element 26 covers a substantial part of the jaws 16, 18 in the axial direction-A. As shown the jaws 16, 18 are connected by at least one resilient member or elastic element 26 integrally mounted onto the jaws 16, 18 in a region between the base portion 12 and the head portions 20, 22. The said resilient member or elastic element 26 exerts a restoring force on the jaws 16, 18, when the jaws 16, 18 are in spread-apart position allowing the needle 34 received in the bore 14 to extend all the way through the needle tip guard 10. As soon as the tip 36 of the needle 34 moves into the needle tip guard 10 i.e. in a position where the distal end section 24 of the first jaw 16 no longer rests over the needle shaft 38, the first jaw 16 snaps back into its relaxed position due to the restoring force exerted by the said integrated resilient member or elastic element 26. The resilient member or elastic element 26 integrally mounted onto the jaws is adapted to form a partial sidewall such that together with the jaws 16, 18 it defines a chamber 46 surrounding the needle 34 in which the needle tip 36 is held after complete withdrawal of the needle 34 from the medical device once the said jaws 16, 18 snaps together protecting the needle tip 36.

As can be seen in FIG. 1, the dimension of the inner portion 28 of the bore in the region adjacent the region of integrally mounted resilient member or elastic element 26 is larger than the dimension inner portion 28 in the region of proximal and distal section of the bore 14. The larger dimension of the inner portion 28 of the bore 14 provides a drawing force for the jaws 16, 18 resulting in improving the restoring force to be exerted by said jaws 16, 18. It is to be noted, however, that the dimension of the inner portion 28 of the bore 14 can have any other suitable dimension or can be uniform through-out extending generally in an axial direction-A.

The jaws 16, 18 are slightly angled towards each other in the region of the resilient member or elastic element 26, resulting in the outer profile of the needle tip guard 10 being tapered towards the distal head portions 20, 22. The outer surface of the jaws 16, 18 may comprises one or more annular protrusions being formed therein. It is to be noted, however, that the needle tip guard can be made without having such annular protrusions.

Further, a locking shoulder 30 is formed at the side of each head portions 20, 22 forming a recess or groove 32 adjacent the distal region of integral resilient member or elastic element 26. One or more locking means are provided in the inner surface of the catheter housing 42 such as an annular locking projection 44 as shown in FIGS. 8 and 9. The annular locking projection 44 may have a profile with rounded edges or substantially rectangular edges or any other profile suitable for locking of the needle tip guard 10 in the catheter housing 42. Further, such locking projection 44 may have continuous annular shape or may have one or more interruptions in between. The housing 42 comprises a main body 50 of generally tubular form and extending in an axial direction-A. The main body has a distal end 52 and a proximal end 54. A catheter 48 is attached to the main body 50 at the distal end 52 of the main body 50. A port 56 extends from the main body 50 in a direction generally perpendicular to the axial direction A. Wings 58 are provided at the main body 50 opposite from the port 56. The main body 50 defines a chamber forming the housing 42 extending from the proximal end 54 towards the distal end 52.

When the needle tip guard 10 is positioned in the catheter housing allowing the needle 34 to extend all the way through the needle tip guard 10 and the catheter housing, the annular locking projection 44 is received in the recess 32 defined by the locking shoulder 30 in the spread-apart position thereby securing the needle tip guard 10 in the catheter housing 42. Preferably, in such a situation, the needle tip guard 10 cannot be pulled out against or relative to the movement of the needle 34 body since the locking shoulder 30 engages with the annular locking projection 44 provided in the catheter housing 42. Thus, as long as the jaws 16, 18 are in a spread-apart position, the needle tip guard 10 is secured against the axial movement relative to the needle 34 body of the catheter device, i.e. the needle tip guard 10 remains steadily in the catheter housing 42. However, when the first jaw 16 adopts its relaxed position as discussed above, the locking protrusion is released from the said recess 32. As a result, the needle tip guard 10 is free to be moved protecting the tip 36 of the needle 34.

Further, it is to be noted that one or more locking and/or releasing means may be provided both inside and/or outside of the said catheter housing 42 for engaging and/or releasing the said needle guard 10 (FIG. 9). Likewise, the outer surface of the needle tip guard 10 may be provided with one or more locking and/or releasing means capable of being in a locking and/or releasing arrangement with the catheter housing 42. For example, the base portion 12 of the needle tip guard 10 may be provided with one or more arms (not shown) having locking means for example locking protrusions at distal ends of either of said arms and/or both the arms to interact with a corresponding recess or projection formed at the outside surface of the catheter housing 42.

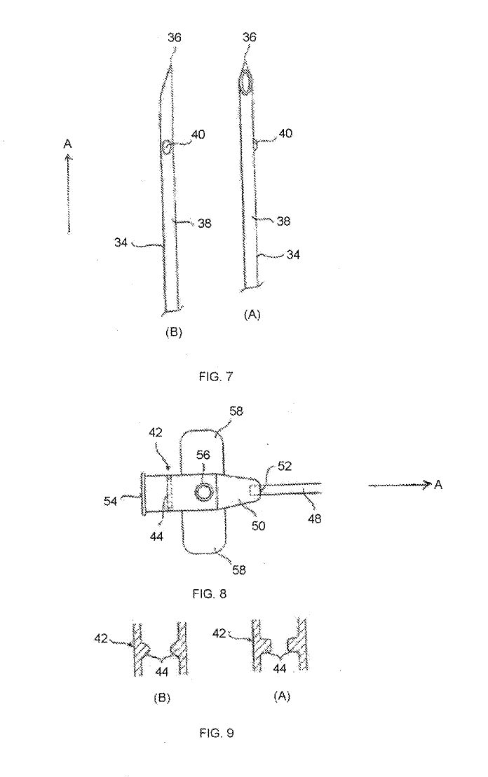

As can be seen in FIG. 7, the needle 34 has a needle tip 36 and a needle shaft 38. The needle shaft 38 has a principal outer profile and defines an axial direction-A. The needle 34 near the tip 36 of the needle is provided with an enlargement 40. The maximum outer dimension/profile of the said enlargement 40 is greater than the principal outer dimension/profile of the needle shaft 38. The position of the enlargement 40 formed on the outer surface of the needle shaft 38 is selected such that the enlargement 40 abuts the base portion 12 of the needle tip guard 10 as soon as first jaw 16 of the needle tip guard 10 no longer rests over the needle shaft 38 in the spread-apart position. Since the maximum outer dimension/profile of the needle 34 in the region of the enlargement 40 is larger than the dimension of the bore 14 in the base portion 12, the needle 34 cannot be further pulled out of the needle tip guard 10. More specifically, because of the said enlargement 40, the needle tip guard 10 cannot slide off the needle 34 during normal use once the needle guard 10 is in a protective position protecting the needle tip 36, unless an excessive external force is applied to the needle 34 and/or needle tip guard 10.

The enlargement 40, for example, can be formed as a crimp by crimping of the needle shaft 38 or as bump with or without addition of additional material by subjecting the needle 34 to concentrated heat at a localized area, for example, using a welding process or a laser welding process. However, it is also possible to form the said enlargement 40 by dispensing additional material onto the outer surface of the needle shaft 38, for example, by welding, soldering or by use of adhesive or glue. Although, only one enlargement 40 is shown in Figs, it is to be noted that more than one enlargement 40 may be present on the outer surface of the needle shaft 38.

Thus, the needle tip guard 10 with the integrated resilient member or elastic element 26 prevents the jaws 16, 18 of the needle tip guard 10 from becoming loose when the needle tip guard 10 slides along the needle 34, thereby aiding and ensuring a correct functioning of the needle tip guard 10. Further, the integrated resilient member or elastic element 26 prevents the needle tip 36 protruding and/or slipping sideways out of the needle tip guard 10, thereby increasing the protection function of the needle tip guard 10.

The integrated resilient member or elastic element 26 is made from a resilient material or elastic material having elastic properties such as, not being limited to, rubber, silicone or the like. Due to its elastic properties, the resilient member or elastic element or ring creates a restoring force on the jaws 16, 18, when the needle 34 fully extends through the needle tip guard 10 thereby spreading the jaws 16, 18 apart and expanding the resilient member or elastic element 26. As soon as the needle tip 36 distal end section 24 of the first jaw 16 upon pulling the needle 34 through the needle safety device 10, the jaws 16, 18 are positively collapsed by the integrated resilient member or elastic element 26 due to its restoring force securing the tip 36 of the needle 34.

For the purpose of a simplified and cost-effective production of the needle tip guard 10, the integrated resilient member or elastic element 26 is may be formed integrally onto the jaws, for example, by injection and/or insert moulding. The resilient member or elastic element 26 may also be configured integrally onto the jaws 16, 18 by other suitable methods such as by use of an adhesive or glue. Further, for a simplified and cost-effective production of the needle tip guard 10, the base portion 12, the resilient member or elastic element 26 and the jaws 16, 18 may be formed from a plastic material, for example, by injection moulding. Alternatively, the base portion 12 and the jaws 16, 18 may be formed from a metal material or combination of materials, such as a different plastic material, a different metal material or a different combination of plastic and/or metal materials. For example, one of the jaws 16, 18 may be made of a metal material and other one 16, 18 may be made of plastic material. Likewise, the base portion 12 may be made from a metal material and the jaws 16, 18 may be made from a plastic material or vice versa. It is also to be noted that the inner part of the jaws 16, 18, which contacts the needle shaft 38, is made from a thermoplastic material such as TPE, whereas the outer part of the jaws 16, 18 may be made from a different material, for example, a plastic, metal, composite or elastomer material, so that the needle tip guard 10 causes less friction when sliding along the needle thereby facilitating the withdrawal of the needle 34.

In various embodiments of the present invention the integrated resilient member or elastic element 26 may comprise a ring like integrated form/structure partially or fully surrounding the jaws 16, 18, and/or clamp, bracket, “C” clip or the like surrounding the jaws 16, 18 only in part.

Although, the invention has been described with reference to certain specific embodiments and examples, it would be appreciated by those skilled in the art that the invention may be embodied in many forms without departing from the broader spirit and scope of the invention as set forth in the invention. Thus, variations of preferred embodiments as disclosed may become apparent to those of ordinary skill in the art upon reading the foregoing description.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. The specification and drawings, therefore, are to be regarded in an illustrative rather than a restrictive manner without departing from the spirit and scope of the invention, the invention resides in the claims hereinafter appended.

1. A needle tip guard for a medical device, in particular for an intravenous catheter assembly, the needle tip guard comprising:
a base portion having a bore extending along an axial direction A for receiving a needle having a needle shaft such that the needle may extend through the bore and move relative to the needle tip guard from a spread-apart position in which the needle tip is outside the needle tip guard to a protective position in which the needle tip is covered by the needle tip guard;
first and second jaws extending from the distal side of said base portion generally in the axial direction A;
each of said first and second jaws having a head portion in the region of its free end, wherein said first jaw rests over the needle shaft in the spread-apart position;
one or more locking means onto the outer surface of the needle tip guard for securing said needle tip guard to said medical device; and
at least one resilient member or elastic element integrally mounted onto said jaws in a region between said base portion and the head portions such that said resilient member or elastic element exerts an inward restoring force on said jaws, when the jaws are fully or partially spread apart by said needle shaft.

2. The needle tip guard according to claim 1, wherein said locking means comprise one or more locking shoulder forming a recess or groove.

3. The needle tip guard according to claim 1, wherein said resilient member or elastic element forms a partial sidewall such that together with the jaws it defines a chamber and is made from a material having elastic properties.

4. The needle tip guard according to claim 1, wherein said bore has a dimension being adapted to the shape and size of the principal outer profile of the needle such that the needle may extend through the bore and move relative to the needle tip guard to a protective position in which the needle tip is covered by said needle guard.

5. The needle tip guard according to claim 1, wherein said first head portion has a distal end section at its free end which extends beyond the second head portion and said distal end section thereof extends towards said second jaw generally perpendicular to the axial direction A.

6. The needle tip guard according to claim 5, wherein the length of said distal end section is as such that the end section protrudes over at least a part of said second head portion.

7. The needle tip guard according to claim 1, wherein said resilient member or elastic element is mounted to mounting features comprising mounting projections and or mounting recesses or the like provided on each of said jaws.

8. The needle tip guard according to claim 1, wherein said resilient member or elastic element further comprises a ring like integrated form/structure partially or fully surrounding the jaws, and/or clamp, bracket, “C” clip or the like surrounding the jaws only in part.

9. An intravenous catheter assembly comprising: a catheter tube, a catheter hub/housing defining a chamber, a needle having a needle tip at the distal end, a needle shaft and a needle hub, and a needle tip guard in particular according to claim 1 slidably arranged on the needle for protecting the needle tip which is housed partially and/or completely in said catheter housing such that the distal end section of the first jaw rests over said needle shaft in the spread-apart position.

10. The intravenous catheter assembly according to claim 9, wherein the inner and/or outer surface of said catheter housing comprises one or more locking and/or releasing means in the form of one or more projections and/or recesses.

11. An intravenous catheter assembly comprising: a catheter tube, a catheter hub/housing defining a chamber, a needle having a needle tip at the distal end, a needle shaft and a needle hub, and a needle tip guard in particular according to claim 8 slidably arranged on the needle for protecting the needle tip which is housed partially and/or completely in said catheter housing such that the distal end section of the first jaw rests over said needle shaft in the spread-apart position.

12. An intravenous catheter assembly comprising: a catheter tube, a catheter hub/housing defining a chamber, a needle having a needle tip at the distal end, a needle shaft and a needle hub, and a needle tip guard in particular according to claim 7 slidably arranged on the needle for protecting the needle tip which is housed partially and/or completely in said catheter housing such that the distal end section of the first jaw rests over said needle shaft in the spread-apart position.

13. An intravenous catheter assembly comprising: a catheter tube, a catheter hub/housing defining a chamber, a needle having a needle tip at the distal end, a needle shaft and a needle hub, and a needle tip guard in particular according to claim 6 slidably arranged on the needle for protecting the needle tip which is housed partially and/or completely in said catheter housing such that the distal end section of the first jaw rests over said needle shaft in the spread-apart position.

14. An intravenous catheter assembly comprising: a catheter tube, a catheter hub/housing defining a chamber, a needle having a needle tip at the distal end, a needle shaft and a needle hub, and a needle tip guard in particular according to claim 5 slidably arranged on the needle for protecting the needle tip which is housed partially and/or completely in said catheter housing such that the distal end section of the first jaw rests over said needle shaft in the spread-apart position.

15. An intravenous catheter assembly comprising: a catheter tube, a catheter hub/housing defining a chamber, a needle having a needle tip at the distal end, a needle shaft and a needle hub, and a needle tip guard in particular according to claim 4 slidably arranged on the needle for protecting the needle tip which is housed partially and/or completely in said catheter housing such that the distal end section of the first jaw rests over said needle shaft in the spread-apart position.

16. An intravenous catheter assembly comprising: a catheter tube, a catheter hub/housing defining a chamber, a needle having a needle tip at the distal end, a needle shaft and a needle hub, and a needle tip guard in particular according to claim 3 slidably arranged on the needle for protecting the needle tip which is housed partially and/or completely in said catheter housing such that the distal end section of the first jaw rests over said needle shaft in the spread-apart position.

17. An intravenous catheter assembly comprising: a catheter tube, a catheter hub/housing defining a chamber, a needle having a needle tip at the distal end, a needle shaft and a needle hub, and a needle tip guard in particular according to claim 2 slidably arranged on the needle for protecting the needle tip which is housed partially and/or completely in said catheter housing such that the distal end section of the first jaw rests over said needle shaft in the spread-apart position.
